(19) United States
(12) Patent Application Publication
(10) Pub. No.: US 2025/0111892 A1
(43) Pub. Date: Apr. 3, 2025

(54) COPY NUMBER VARIANT DETECTION

(71) Applicant: INTEGRATED DNA TECHNOLOGIES, INC., Coralville, IA (US)

(72) Inventors: Ryan Rogge, Coralville, IA (US); Taylor Patterson, Coralville, CO (US); Allison Hadjis, Coralville, IA (US); Mark Rogers, Coralville, IA (US); Christina Cleveland, Coralville, IA (US); Morgan Weichert, Coralville, IA (US)

(21) Appl. No.: 18/899,806

(22) Filed: Sep. 27, 2024

Related U.S. Application Data
(60) Provisional application No. 63/586,051, filed on Sep. 28, 2023.

(51) Int. Cl. G16B 20/10

(52) U.S. Cl. CPC G16B 20/10

(57) ABSTRACT

Described herein are methods for determining the number of unique sequence molecules, such as copy number variants and breakpoints, in Anchored Multiplex PCR (AMP) panels using only sequencing data from the sample of interest.

### CROSS-REFERENCE TO RELATED APPLICATIONS

This application claims priority to U.S. Provisional Patent Application No. 63/586,051, filed on Sep. 28, 2023, which is incorporated by reference herein in its entirety.

### BACKGROUND

Copy number variants (CNVs), also referred to as Copy Number Alteration or Aberration (CNA), describe instances where the number of copies of a region of the genome differs from the expected number (e.g., usually two copies expected in humans). Errors in DNA replication, repair, or recombination, and other processes can cause CNVs. CNVs may be a cause of disease, a symptom, or both. Copy gains or losses affecting oncogenes or tumor suppressor genes are one mechanism by which cancers may arise, proliferate, or persist. CNVs may be targetable by, or grant resistance to, certain therapies. Patterns of CNV may signal chromosomal instability resulting from homologous recombination deficiency (HRD).

Current CNV methods require a control baseline or reference to compare against and calculate fold change values, which usually involves matched/paired normal tissue or a cohort/panel of representative normal samples. Some next-generation sequencing (NGS) methods allow internal, self-normalization methods, but this is typically only used with whole genome sequencing data.

What is needed are new methods for determining CNVs and breakpoints in Anchored Multiplex PCR (AMP) panels using only data from the sample of interest.

### SUMMARY

One embodiment described herein is a computer-implemented method, the method comprising: receiving, by one or more processors, data representative of one or more primer counts; identifying, by one or more processors, whether the data representative of any one of the one or more primer counts is suitable to be fit into a regression model; transferring into the regression model, by one or more processors, the data representative of the one or more suitable primer counts; determining, by one or more processors, one or more segments associated with the data representative of the one or more suitable primer counts by applying kernel change point detection to the data representative of the one or more suitable primer counts; and determining, by one or more processors, a fold change between the one or more segments relative to a baseline measure, wherein the fold change is representative of a quantity of unique molecules. In one aspect, the method further comprises: annotating, by one or more processors, the data representative of the one or more suitable primer counts with a value representative of an asymptote for the data. In another aspect, the method further comprises: removing by covariate correction, by one or more processors, all primer counts which are not suitable to be fit into the regression model; and applying guanine-cytosine (GC) bias correction, by one or more processors, to the asymptotes of the one or more suitable primer counts. In another aspect, the GC bias correction is locally weighted scatterplot smoothing (LOWESS). In another aspect, the regression model is a Michaelis-Menten (MM) model.

Another embodiment described herein is a computer program product, the computer program product comprising: one or more computer-readable storage media and program instructions stored on the one or more computer-readable storage media, the program instructions comprising: program instructions to receive data representative of one or more primer counts; program instructions to identify whether the data representative of any one of the one or more primer counts is suitable to be fit into a regression model; program instructions to transfer into the regression model the data representative of the one or more suitable primer counts; program instructions to determine one or more segments associated with the data representative of the one or more suitable primer counts by applying kernel change point detection to the data representative of the one or more suitable primer counts; and program instructions to determine a fold change between the one or more segments relative to a baseline measure, wherein the fold change is representative of a quantity of unique molecules. In one aspect, the program instructions further comprise: program instructions to annotate the data representative of the one or more suitable primer counts with a value representative of an asymptote for the data. In another aspect, the program instructions further comprise: removing by covariate correction, by one or more processors, all primer counts which are not suitable to be fit into the regression model; and applying guanine-cytosine (GC) bias correction, by one or more processors, to the asymptotes of the one or more suitable primer counts. In another aspect, the GC bias correction is locally weighted scatterplot smoothing (LOWESS). In another aspect, the regression model is a Michaelis-Menten (MM) model.

Another embodiment described herein is a computer system, the computer system comprising: one or more processors; one or more non-transitory computer-readable storage media; and program instructions stored on at least one of the one or more non-transitory computer-readable storage media for execution by at least one of the one or more processors, the program instructions comprising steps for implementing the following acts: program instructions to receive data representative of one or more primer counts; program instructions to identify whether the data representative of any one of the one or more primer counts is suitable to be fit into a regression model; program instructions to transfer into the regression model the data representative of the one or more suitable primer counts; program instructions to determine one or more segments associated with the data representative of the one or more suitable primer counts by applying kernel change point detection to the data representative of the one or more suitable primer counts; and program instructions to determine a fold change between the one or more segments relative to a baseline measure, wherein the fold change is representative of a quantity of unique molecules. The computer system of clause 11, the system further comprises: program instructions to annotate the data representative of the one or more suitable primer counts with a value representative of an asymptote for the data. In another aspect, the system further comprises: removing by covariate correction, by one or more processors, all primer counts which are not suitable to be fit into the regression model; and applying guanine-cytosine (GC) bias correction, by one or more processors, to the asymptotes of the one or more suitable primer counts. In another aspect, the GC bias correction is locally weighted scatterplot smoothing (LOWESS). In another aspect, the regression model is a Michaelis-Menten (MM) model.

### DESCRIPTION OF THE DRAWINGS

FIG. 1 shows a block diagram of an exemplary computing environment 100, according to some implementations of the present disclosure.

FIG. 2 shows a block diagram of a controller included in the exemplary computing environment system of FIG. 1, according to some implementations of the present disclosure.

FIG. 3 shows a flowchart of an exemplary process 300 to determine a fold change between one or more segments relative to a baseline measurement, according to some implementations of the present disclosure.

FIG. 4A-B show example diagrams illustrating CNV gains and losses, allele-specific copy numbers (ASCN), loss of heterozygosity (LOH), and copy-neutral LOH. FIG. 4A shows example fold change diagrams using primer counts for normal copy numbers, a CNV gain, and a CNV loss.

FIG. 4B shows example fold change and allele-frequency (AF) diagrams using primer counts for normal copy numbers, a CNV allele-specific gain, a CNV LOH, and a copy-neutral LOH.

FIG. 5 shows a non-limiting example process workflow for a cohortless CNV detection method as described herein. Step 1—Subsampling of primer counts with a .map file. Step 2—Model fitting to subsampled primer counts to create asymptote. Step 3—Data curation: model fit quality control, GC % correction, outlier removal. Step 4—Segmentation of asymptote values using kernel change point detection. Step 5—Identify baseline by randomly sampling filtered asymptotes from non-sex chromosomal positions many times. Step 6—Statistically test segments against the baseline for significance and calculate fold change relative to baseline.

FIG. 6A-C show example plots for the different steps of a cohortless CNV detection method as described herein. FIG. 6A shows example plots of subsampling primer counts (i.e., rarefaction) and model fitting for chromosomes 6 and 7. FIG. 6B shows example plots of GC % bias correction (pre-covariate and post-covariate corrections). FIG. 6C shows example plots of segment-corrected asymptotes for baseline determination for chromosomes 6 and 7.

FIG. 7A-D show event detection results of a conventional Archer® Analysis CNV method using three different sample size cohorts (FIG. 7A-C), as compared to a cohortless CNV method as described herein (FIG. 7D), for the same sample library.

FIG. 8A-C show results from an exemplary cohortless CNV process as described herein. Results are shown for CNV breakpoint detection of synthetic MET amplification in challenging low input mass and contrived low aberrant cellularity libraries. VARIANT Plex® libraries were prepared using 10 ng total input mass of either Seraseq® FFPE WT DNA reference material (FIG. 8A), Seraseq® Compromised FFPE Tumor DNA reference material (FIG. 8C), or a mix of 30% Tumor DNA reference material with the WT DNA reference material as background (FIG. 8B). Libraries were then sequenced and analyzed by the cohortless CNV process in Archer® Analysis.

FIG. 9 shows results obtained using an exemplary cohortless CNV method as described herein compared to digital drop PCR (ddPCR) methods for determining the abundance of specific sequences. Results are shown for ddPCR and a cohortless CNV method for 108 unique inputs, including tumor FFPE extracts, cell lines, and reference materials, with a variety of amplifications and deletions. The x and y values are the ratios (or mean ratios when available) calculated from ddPCR concentrations or CNV segment fold changes for segments that overlap with the coordinates of the ddPCR probes used. Standard deviation error bars are drawn, and mean ratio values plotted when there were replicate libraries, replicate ddPCR results, or both.

FIG. 10A-D show results demonstrating the capability of the disclosed cohortless CNV method to identify and call a variety of CNV events in different applications. FIG. 10A shows a homozygous deletion of SMN2 on chromosome 5 that was detected. FIG. 10B shows a heterozygous loss of chromosome X in XY samples that was detected. FIG. 10C shows a whole chromosome gain of chromosome 8 that was detected. FIG. 10D shows very large copy number gains detected in MYCN and exons 3 and 4 of ALK on chromosome 2 in a neuroblastoma cell line.

FIG. 11 shows a receiver operating characteristic (ROC) curve plot of sensitivity and specificity, demonstrating the overall functionality of counts through the cohortless CNV process described herein (“Std Cohortless”). These results were compared to two internal NGS processes on the same data (“Normal Analysis” and “Unique Counts”).

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ˜ 20° C., ˜22° C., ˜25° C., or ˜27° C., at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

Systems, methods, and techniques are disclosed herein for cohortless copy number variation (“CNV”) modeling that is applied to determine a CNV call per primer segment of a sample. As used herein, “cohortless” means that a normal dataset, panel of normals, etc. is not required. In some embodiments described herein, regions of similar copy number are automatically segmented together. Segmentation provides “groups” without a priori knowledge of CNV extent or breakpoints. In contrast to current conventional techniques which utilize deduplicated read counts, the cohortless CNV methods described herein compensate for sampling bias and estimate how saturated the deduplicated (unique) counts are. This allows for an improved calculation of how abundant the underlying sequences are in the library.

Copy number variants can be drivers of disease (e.g., loss of a tumor suppressor gene, gain in an oncogene). Copy number variants can also be symptoms of disease (e.g., accumulation of CNVs in homologous recombination deficient (HRD) cases). Copy number variants can inform prognosis, progression, and therapy selection.

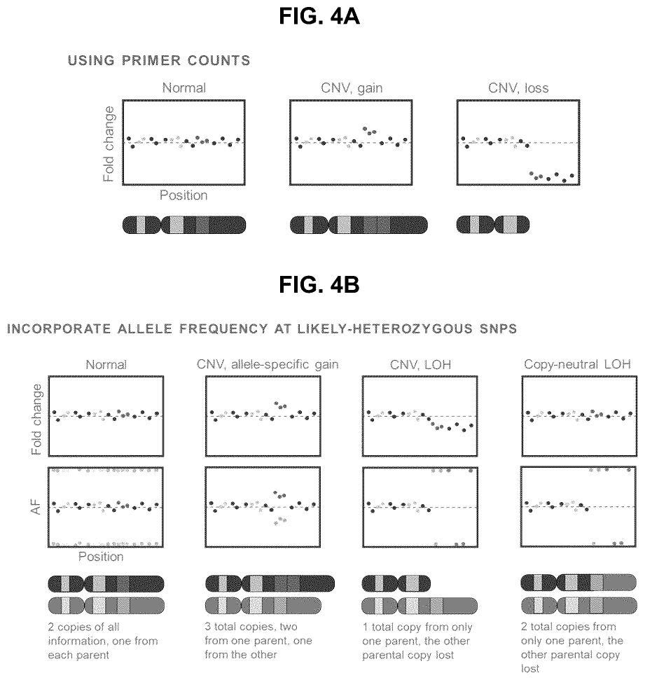

FIG. 4A-B show example diagrams illustrating CNV gains and losses, allele-specific copy numbers (ASCN), loss of heterozygosity (LOH), and copy-neutral LOH. FIG. 4A shows example fold change diagrams using primer counts for normal copy numbers, a CNV gain, and a CNV loss. FIG. 4B shows example fold change and allele-frequency (AF) diagrams using primer counts for normal copy numbers, a CNV allele-specific gain, a CNV LOH, and a copy-neutral LOH. As used herein, the term “primer counts” refers to the number of raw and deduplicated (unique) reads attributed to a primer.

In one aspect, the described methods may be used to calculate copy number variations from NGS data (particularly AMP libraries) without the need for comparison of counts to a normal sample or representative cohort of normal samples (i.e., cohortless detection). In addition, while the disclosed methods make it possible to calculate copy number variations from NGS data without having to compare to a normal dataset, the disclosed methods still result in increased CNV accuracy when used in workflows that do involve a comparison to a normal dataset.

In another aspect, the described methods may be used for any NGS calculations where the abundance of a DNA fragment is important to downstream calculations. RNA expression analysis and pathogen abundance analysis by NGS are other types of NGS analyses where this underlying approach of counting would lead to improvements.

In another aspect, the described methods relate to computing the number of DNA fragments in an NGS library based on the data from an NGS sequencer. Currently, conventional methods rely on deduplicating sequences based on unique molecular indexes (UMI) and utilize that deduplicated count. In some embodiments described herein, the disclosed methods involve rarefaction of UMI data and extrapolation to saturation, to predict the number of deduplicated molecules if an infinite amount of sequencing were performed. In some embodiments, the disclosed methods minimize the number of NGS reads necessary to accurately assess the abundance of particular DNA fragments in an NGS library.

As will be appreciated by one skilled in the art, the methods and systems described herein may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the methods and systems may take the form of web-implemented computer software. Any suitable computer-readable storage medium may be utilized including, but not limited to, hard disks, CD-ROMs, optical storage devices, or magnetic storage devices.

Embodiments of the methods and systems are described below with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions, and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

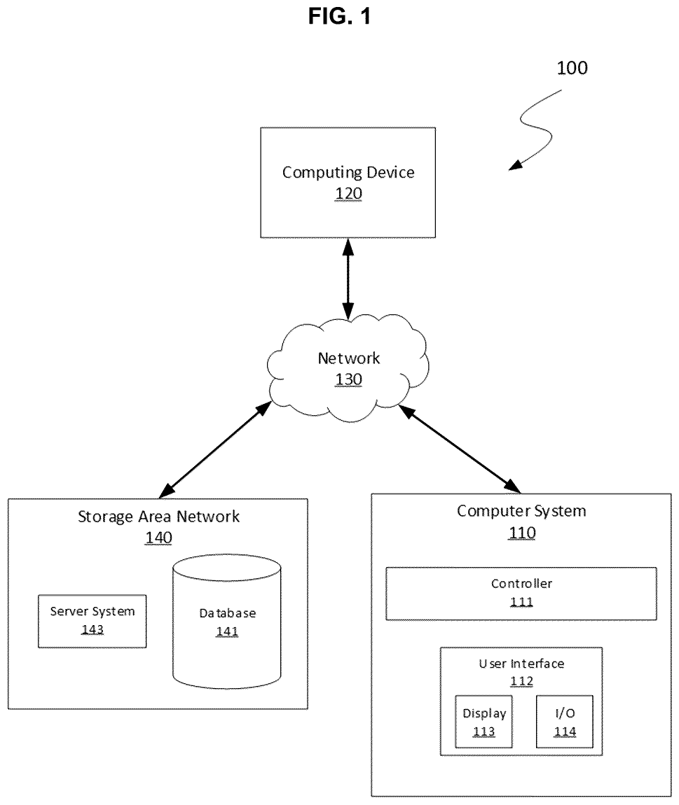

FIG. 1 illustrates a computing environment 100. As illustrated in FIG. 1, the computing environment 100 includes computer system 110, computing device 120, and storage area network 140 connected over network 130. Computer system 110 includes controller 111, user interface 112, display 113, and I/O 114. Storage area network (SAN) 140 includes database 141 and server system 143.

In various embodiments, computer system 110 is a computing device that can be a standalone device, a server, a laptop computer, a tablet computer, a netbook computer, a personal computer (PC), a personal digital assistant (PDA), a desktop computer or any programmable electronic device capable of receiving, sending, and processing data. In general, computer system 110 represents any programmable electronic device or combination of programmable electronic device capable of executing machine readable program instructions and communication with SAN 140 and computing device 120. In another embodiment, computer system 110 represent a computing system utilizing clustered computers and components to act as a single pool of seamless resources. In general, computer system 110 can be any computing device or a combination of devices with access to SAN 140, computing device 120, and network 130 and is capable of executing controller 111, user interface 112, display 113 and I/O 114. Computer system 110 may include internal and external hardware components, as depicted and described in further detail with respect to FIG. 1.

In one aspect, controller 111, user interface 112, display 113, and I/O 114 are stored on computer system 110. However, in another aspect, controller 111, user interface 112, display 113, and I/O 114 may be stored externally and accessed through a communication network, such as network 130. Network 130 can be, for example, a local area network (LAN), a wide area network (WAN) such as the Internet, or a combination of the two, and may include wired, wireless, fiber optic, or any other connection known in the art. In general, network 130 can be any combination of connections and protocols that will support communications between computer system 110, computing device 130, and SAN 140, in accordance with a desired embodiment of the present invention.

In one aspect, user interface 112 operates on computer system 110. User interface 112 provides an interface between computer system 120 and SAN 140. In another aspect, user interface 112 can be a graphical user interface (GUI) or a web user interface (WUI) and can display text, documents, web browsers, windows, user options, application interfaces, and instructions for operation, and includes the information (such as graphic, text, and sound) that a program presents to a user and the control sequences the user employs to control the program. In another aspect, computer system 110 accesses data stored on computing device 130 and/or SAN 140 via a client-based application that runs on computer system 110. For example, computer system 110 includes mobile application software that provides an interface between computer system 110, computing device 130, and SAN 140.

SAN 140 is a storage system that includes database 141 and server system 143. SAN 140 may include one or more, but is not limited to, computing devices, servers, server-cluster, web servers, database, or storage devices. SAN 140 operates to communicate with computer system 110 and/or computing device 130, and various other computing systems and/or devices (not shown) over a network, such as network 130. For example, SAN 140 communicates with controller 111 to transfer data between, but is not limited to, database 141 and various other databases (not shown) that are connected over network 130. In general, SAN 140 can be any computing device or combination of devices that are communicatively connected to a local IoT network, i.e., a network comprised of various computing systems and sensory devices including but are not limited to computer system 110 and/or computing device 130, to provide functionality described herein. SAN 140 can include internal and external hardware components. The present invention recognizes that FIG. 1 may include any number of computing devices, servers, databases and/or storage devices, and the present invention is not limited to only what is depicted in FIG. 1. As such, in another aspect, some or all of the features and functions of SAN 140 are included as part of computer system 110 and/or another computer system. Similarly, in another aspect, some of the features and functions of computer system 110 are included as part of SAN 140 and/or another computer system.

In one aspect, SAN 140 represents a cloud computing platform. Cloud computing is a model of configurable computing resources (e.g., networks, network bandwidth, servers, processing, memory, storage, applications, virtual machines, and services) that can be rapidly provisioned and released with minimal management effort or interaction with a provider of a service. A cloud model may include characteristics such as on-demand self service, broad network access, resource pooling, rapid elasticity, and measured service, can be represented by service models including a platform as a service (PaaS) model, an infrastructure as a service (IaaS) model, and a software as a service (SaaS) model, and can be implemented as various deployment models including as a private cloud, a community cloud, a public cloud, and a hybrid cloud.

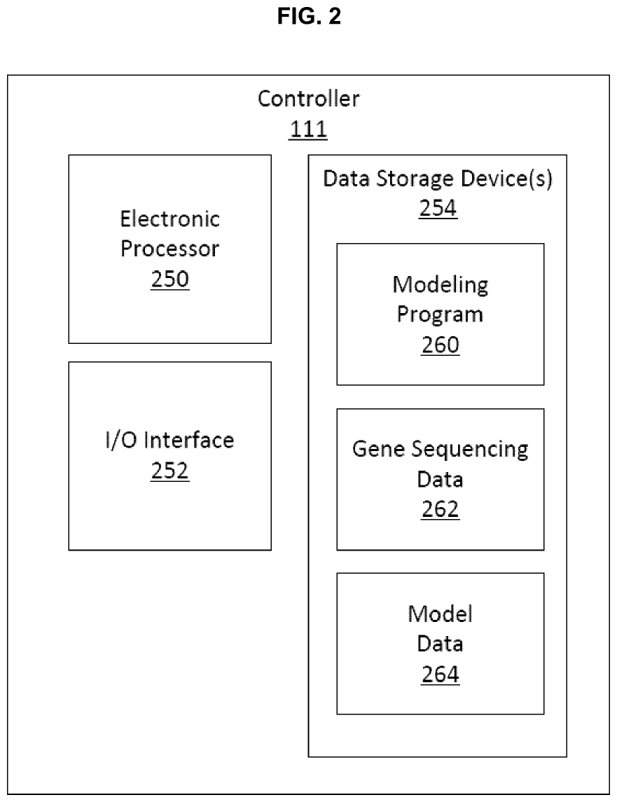

In one aspect, as illustrated in FIG. 2, the controller 111 may include an electronic processor 250, an input/output (I/O) 252, and data storage device 254; however, it should be understood that the controller 111 may have additional or fewer components as suitable for the application and setting, such as, for example, multiple electronic processors, multiple I/O interfaces, multiple data storage devices, or a combination thereof. In another aspect, some or all of the components included in controller 111 may be attached to one or more motherboards and enclosed in a housing (e.g., including plastic, metal, and/or other materials). In another aspect, some of these components may be fabricated onto a single system-on-a-chip (SoC) (e.g., an SoC may include one or more processing devices and one or more storage devices).

As used herein, “processor” or “electronic processor” refers to any device or portion of a device that processes electronic data from registers and/or memory to transform that electronic data into other electronic data that may be stored in registers and/or memory. The electronic processor 250 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

The data storage device 254 may include one or more memory devices such as random-access memory (RAM) devices (e.g., statis RAM (SRAM) devices, magnetic RAM (MRAM) devices, dynamic RAM (DRAM) devices, resistive RAM (RRAM) devices or conductive-bridging RAM (CBRAM) devices), hard drive-based memory devices, solid-state memory devices, networked drives, cloud drives, or any combination of memory devices. In one aspect, the data storage device 254 may include memory that shares a die with a processor. The memory may be used as cache memory and may include embedded dynamic random-access memory (eDRAM) or spin transfer torque magnetic random-access memory (STT-MRAM), for example. The data storage device 254 may include non-transitory computer readable media having instructions thereon that, when executed by one or more processors (e.g., the electrical processor 250), causes the controller 111 to perform any appropriate ones or portions of the methods disclosed herein. For example, one or more data storage devices 254 included in the controller 111 may store various applications and data for performing one or more of the methods described herein or portions described herein. For example, the one or more data storage devices 254 may store modeling program 260, gene sequencing data 262, and model data 264. It should be understood that each method described herein may be implemented via one application or multiple applications.

The I/O interface 252 of the controller 111 may include one or more communication chips, connectors, and/or other hardware and software to govern communications between the controller 111 and other components. The I/O interface 252 may include interface circuitry for coupling to the one or more components using any suitable interface (e.g., a Universal Serial Bus (USB) interface, a High-Definition Multimedia Interface (HDMI) interface, a Controller Area Network (CAN) interface, a serial Peripheral Interface (SPI) interface, an Ethernet interface, a wireless interface, or any other appropriate interface). For example, I/O interface 252 may include circuitry for managing wireless communications for the transfer of data to and from the controller 111. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although in some embodiments they might not. Circuitry included in I/O interface 252 for managing wireless communications may implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any embodiments, updates, and/or revisions (e.g., advanced LTE project, ultra-mobile broadband (UMB) project (also referred to as “3GPP2”), etc.). Circuitry included in the I/O interface 252 for managing wireless communications may operate in accordance with a Global System for Mobile Communication (GSM), General Packet Radio Service (GPRS), Universal Mobile Telecommunications System (UMTS), High0-Speed Packet Access (HSPA), Evolved HSPA (E-HSPA), or LTE network. Circuity may also be included in the I/O interface 252 for managing wireless communications, which may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). Circuitry included in the I/O interface 252 for managing wireless communications may operate in accordance with Code Division Multiple Access (CDMA), Time Division Multiple Access (TDMA), Digital Enhanced Cordless Telecommunications (DECT), Evolution Data Optimized (EV-DO, and derivatives thereof, as well as any other wireless protocols that are designated as 3G, 4G, 5G, and beyond. The I/O interface 252 may include one or more antennas (e.g., one or more antenna arrays) to receipt and/or transmission of wireless communications.

In one aspect, the modeling program 260 may be configured to operate on the computer system 110. The modeling program 260 may be configured to access, retrieve, receive, identify, analyze, determine, and/or generate data stored on the gene sequencing data 262. In another aspect, the modeling program 260 may be configured to operate on a computing device (e.g., computing device 120) communicatively connected (e.g., network 130) to computer system 110.

In one aspect, the gene sequencing data 262 may include data representative of one or more molecular barcodes associated with a sample of a genome. The one or more molecular barcodes may be unique molecular indexes (UMI), where the UMIs are short sequences used to uniquely tag each molecule in a sample library. UMIs are used for a wide range of sequencing applications. Sequencing with UMIs can reduce the rate of false-positive variant calls and increase sensitivity of variant detection. Each nucleic acid in the starting material is tagged with a unique molecular barcode, bioinformatics may filter out duplicate reads and PCR errors with a high level of accuracy and report unique reads, removing the identified errors before final data analysis.

In one aspect, the model data 264 may include any number of regression analyses including, non-linear regression models, partial least squares regression (PLS) model, partial least squares discriminant analysis (PLSDA), principal component regression (PCR) model, least absolute shrinkage and selection operator (LASSO) model, elastic-net regression model, support vector machine (SVM) model, neural network model, or combinations thereof stored on the data storage device 254. A brief description of the use of these regression analyses or machine learning processes are described below.

In one aspect, the above-described regression analysis and/or machine learning processes may be implemented on one or more processors. The one or more processors may be operating on the controller 111 and/or a third-party computing device (e.g., computing device 120) communicatively connected to the computer system 110.

In one aspect, the PLS model is a statistical method that generalizes and combines features from principal component analysis and multiple regression. It can be useful to predict a set of dependent variables from a very large set of independent variables (i.e., predictors). The goal of PLS regression is to predict Y and X and to describe their common structure. When Y is a vector and X is full rank, the goal may be accomplished using ordinary multiple regression. When the number of predictors is large compared to the number of observations, X is likely to be singular and the regression approach is no longer feasible (i.e., because of multicollinearity).

In one aspect, the non-linear regression model may be a Michaelis-Menten (MM) model. The MM model provides for enzyme kinetics, where the MM model determines the enzyme's Kₘ (i.e., substrate concentration that yields a half-maximal velocity) and Vₘₐₓ (i.e., maximum velocity). An XY data table is created, where X is representative of raw counts associated with the primer counts and Y is representative of unique counts associated with the primer counts. Vₘₐₓ is the maximum enzyme velocity in the same units as the Y value. It is the velocity of the enzyme extrapolated to an infinite raw count for each primer at each subsampling level.

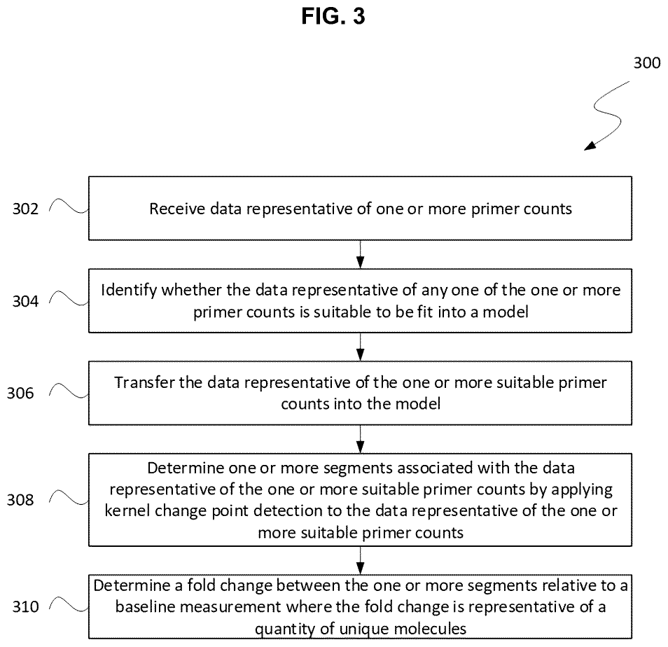

Referring to FIG. 3, a flowchart illustrates an exemplary process 300 for a fold change between one or more segment relative to a baseline measure, in accordance with some implementations of the present disclosure. Process 300 may be implemented using the computer system 110, as described above. The process 300 is described herein as being performed via the controller 111. However, it should be understood that the process 300 may be performed by one or more software and/or hardware components in various combinations and configurations. As illustrated in FIG. 3, the process 300 may include operation 302, 304, 306, 308, and 310. In another aspect, the process 300 is performed in the order as illustrated in FIG. 3. In another aspect, the process 300 may be performed in order other than is illustrated in FIG. 3.

In one aspect, the controller 111 receives data representative of one or more primer counts (operation 302).

In one aspect, the controller 111 identifies whether data representative of any one of the one or more primer counts is suitable to be fit into a regression model (operation 304).

In one aspect, the controller 111 transfers the data representative of the one or more suitable primer counts onto the regression model (operation 306).

In one aspect, the controller 111 determines one or more segment associated with the data representative of the one or more suitable primer counts by applying kernel change point detection to the data representative of the one or more suitable primer counts (operation 308).

In one aspect, the controller 111 determines a fold change between the one or more segments relative to a baseline measure where the fold change is representative of a quantity of a unique molecules (operation 310).

In one aspect, copy number variants (CNVs) describe instances where the number of copies of a region of the genome differs from the expected number. Errors in DNA replication, repair, or recombination, and other processes can cause CNVs. CNVs may be a cause of disease, a symptom, or both. Copy gains or losses affecting oncogenes or tumor suppressor genes are one mechanism by which cancers may arise, proliferate, or persist. CNVs may be targetable by, or grant resistance to, certain therapies. Patterns of CNV may signal chromosomal instability resulting from homologous recombination deficiency (HRD). CNV methods require a baseline to compare against and calculate fold change values, usually matched normal tissue or a panel of normal samples. Some next-generation sequencing (NGS) methods allow internal, self-normalization methods but recommend this only for whole genome sequencing data. As described herein, a new CNV method was introduced which relies only on data from the sample of interest (i.e., cohortless) to determine copy number and breakpoints. The methods described herein are designed to work for Anchored Multiplex PCR (AMP) panels.

In one non-limiting aspect, raw reads from a sequenced AMP library are deduplicated using molecular barcodes incorporated during AMP library preparation and aligned by Archer® Analysis as for any DNA workflow. Read counts for each primer are corrected for GC %, PCR, and sequencing biases. Outliers are removed, then the remaining bias-corrected values are segmented. Baseline copy number is estimated from bias-corrected counts from autosomal primers. Each segment's mean is tested against the estimated baseline copy number and fold changes calculated. CNV classifications can then be made based on p-value.

In another non-limiting exemplary aspect, in development testing with 212 formalin-fixed, paraffin-embedded (FFPE) tissue and cell line reference inputs prepared with panels ranging in size from 499 to 2595 primers, this method can detect single copy gains and losses, homozygous deletions, and aneuploidy, with specific resolution dependent on a panel's primer distribution. In addition to sensitive detection, the disclosed method estimates fold change values that exhibit high agreement with ddPCR fold changes. In one non-limiting example, comparing 142 FFPE samples and cell line reference standards prepared with AMP panels and analyzed by ddPCR yields a concordance correlation coefficient of 0.945. This AMP-based NGS CNV method demonstrates strong performance without requiring paired normal or a panel of normals. These CNV data and results can then be incorporated into further calculations including allele-specific copy number (ASCN) and homologous recombination deficiency (HRD) status determinations. For example, CNV fold change and breakpoint results can be combined with allele frequency data from single nucleotide polymorphisms to determine the contribution of different alleles to the measured CNV fold change. This can enable estimation of absolute total and minor allele copy numbers and identification of events of interest such as losses of heterozygosity (LOH). Additionally, CNV results on their own, or as incorporated ASCN results, can be used to make HRD status classifications by measuring the degree of genomic scarring caused by incorrectly repaired breaks, a consequence of HRD, which often appear as CNV events.

### Methods of Operation

### Pre-Work Overview

To acquire the necessary NGS primer counts data for computational analysis, .map files may be generated using Archer® Analysis and AMP panels. These .map files contain information for the rest of the process. First, the file may contain information regarding the molecular barcode (i.e., unique molecular indexes (UMI)) and how many times it was observed. This information is generated during a process called read deduplication, which uses the molecular barcode to build NGS read bins and consensus reads.

Second, the file contains information from the alignment or mapping process by which the NGS read is associated with a specific location within a reference genome. For other NGS chemistries and pipelines, similar information is needed for the UMI/molecular barcode and mapping. These are common processes for NGS data with UMIs, and these processes and the resulting data are necessary before extrapolation can be performed in the next steps.

### Predicting NGS Unique Counts to Reduce Measurement Bias

The overall goal of this process is to improve the estimate of unique molecule counts for an NGS library of molecules. Molecular barcode subsampling is used to generate multiple data points simulating a fewer number of raw read measurements. The .map file is processed to obtain all unique molecular barcodes, their raw counts, and corresponding primers (if they mapped to a known primer). Subsampling is then performed to multiple lower fractions of raw counts without replacement from all the molecular barcodes to obtain new raw and unique counts per primer at each subsample fraction. This creates a data frame with the raw and unique counts per primer at each subsampling level. In initial testing, sampling without replacement was superior to sampling with replacement. The number of points to subsample to, and whether to perform replicates, were all tested without large impact as long as the subsampled points were greater than 3.

Model curve fitting of subsampled primer counts is then performed to generate an estimate of the number of unique molecules at infinite raw read measurements. This checks whether a primer is suitable for Michaelis-Menten (MM) model fitting based on its total raw counts, total unique subsampling points, and deduplication ratio (unique/raw reads). If a primer is suitable for MM fitting, a non-linear least squares method from scipy can be used to fit the subsampling points to the MM function. If a primer is not suitable, a curve fit is not used and instead the original unique counts are used as the estimated unique counts. In some embodiments, it may be necessary to include data points for nucleic acid sequences that may be absent in solution, e.g., homozygous deletion in genomic DNA. Each primer is then annotated with its model fit results. The primary annotation and data of interest for further processing are the values of the asymptote (maximum) for the curve. Other curve fitting methods besides MM fitting can also be used for suitable fitting. For example, a similar approach could be taken using non-parametric methods for predicting where the data are heading.

Primer cleanup, quality control, and correction are then performed. First, any primers derived from MM fitting that were deemed poor fits based on the relative error of the asymptote estimate are removed. This is performed prior to covariate correction to capture the true quality of the curve fit before it is adjusted by covariate correction. Second, all remaining primer asymptotes are adjusted to account for covariates (other than initial concentration) that could potentially bias the results. In addition, the GC content around each primer may be corrected at this step. Locally weighted scatterplot smoothing (LOWESS) may then be used to fit the asymptotes to the covariates and the asymptotes are then adjusted based on the estimated relationship between the two. Covariate correction comes after filtering to avoid biasing the MM curve fit filters, but before outlier removal, to improve outlier detection. The Vₘₐₓ values may also require correction for GC content even if outlier removal is turned off.

Using Unique Count Predictions for CNV Determination without Comparing to a Normal Reference Data Set

These steps of the process are important to the copy number variation (CNV) portion of the workflow. These steps are separate from the above process which is an improved method for determining unique counts from UMI data for any NGS library data. The following cohortless CNV determination process is an application made possible using the improved unique counts.

Primer segmentation by estimated counts is first performed. The change-point detection algorithm from the main segmentation step (below) is used to identify isolated, single-primer segments for removal. The minimum segment size is reduced to facilitate segmentation of isolated primers. Segmentation is made more permissive to facilitate creation of isolated single-primer segments. Isolated single-primer segments are used as likely local outliers. The Kernel Change Point Detection method is then used to identify groups of adjacent primers with similar counts. This creates initial segmentation results that include aggregate information for each segment identified.

Baseline counts identification is then performed. This pipeline does not employ a normal cohort, so there are no expected baseline counts (i.e., counts for regions in the absence of copy number changes). Instead, a baseline segment is approximated for the sample by bootstrapping (i.e., randomly sampling many times) all of the primers. First, outliers are filtered out (based on an IQR filter) and autosomal (chrX/Y) primers are removed. Next, a mean median count per primer (the average of all the median counts) and an error on the mean median are calculated. This median becomes the starting point for baseline identification since it should be robust to outliers and samples that have ubiquitous copy number changes.

For statistical calling of detected CNVs, aggregate statistics are collected across all segments for the sample. For all segments, the sample average segment information (mean segment length, mean segment Vₘₐₓ, mean segment error) and the adjusted baseline+baseline error from bootstrapping are used to approximate a canonical baseline segment for the particular chromosome. The model baseline segment is used to perform a two-sample t-test with each of the segments from that chromosome. After testing all segments within a chromosome, multi-test correction is then performed. Segments are then annotated with their corrected p-values. Using pre-defined alpha levels, a CNV call per segment is made and the segment can be annotated with its call.

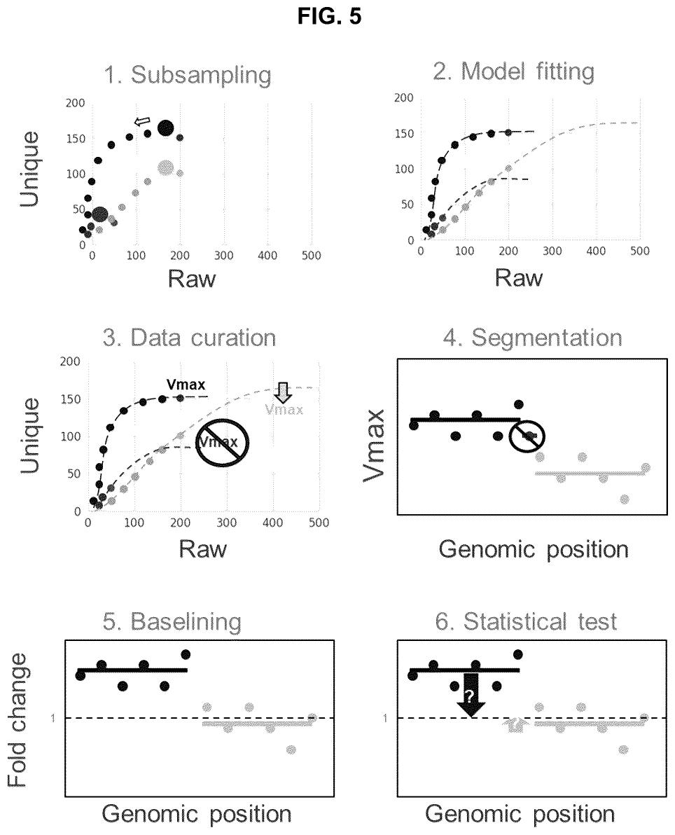

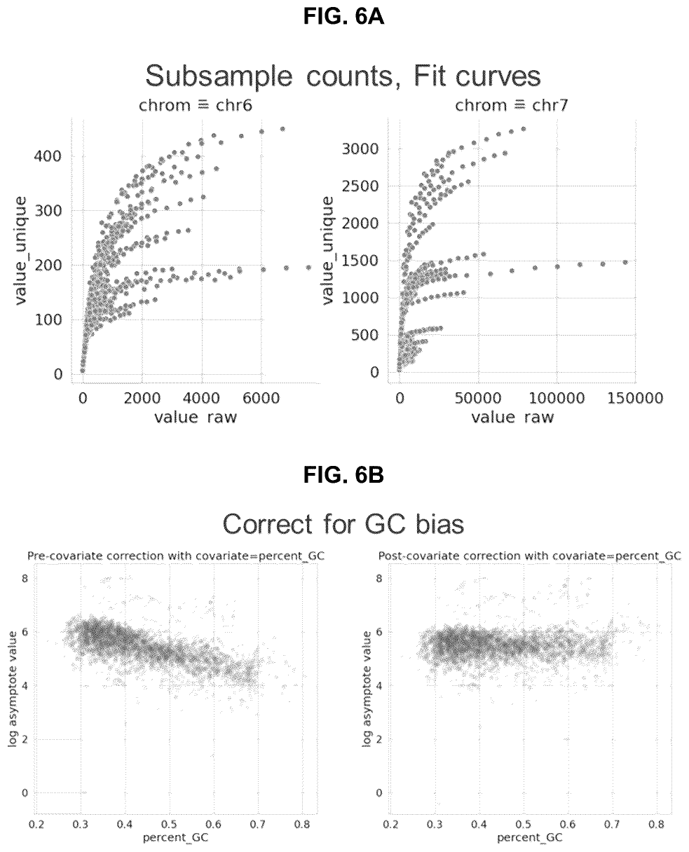

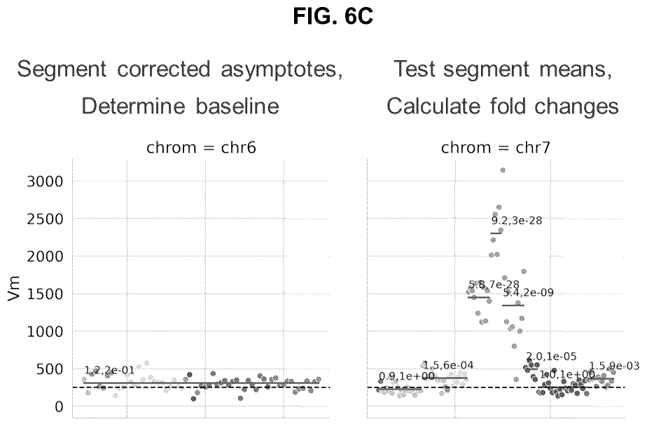

FIG. 5 shows a non-limiting example workflow for this cohortless CNV detection method. Step 1—Subsampling of primer counts with a .map file. Step 2—Model fitting to subsampled primer counts to create asymptote. Step 3—Data curation: model fit quality control, GC % correction, outlier removal. Step 4—Segmentation of asymptote values using kernel change point detection. Step 5—Identify baseline by randomly sampling filtered asymptotes from non-sex chromosomal positions many times. Step 6—Statistically test segments against the baseline for significance and calculate fold change relative to baseline. FIG. 6A-C show example plots for the different steps of a cohortless CNV detection method as described herein. FIG. 6A shows example plots of subsampling primer counts (i.e., rarefaction) and model fitting for chromosomes 6 and 7. FIG. 6B shows example plots of GC % bias correction (pre-covariate and post-covariate corrections). FIG. 6C shows example plots of segment-corrected asymptotes for baseline determination for chromosomes 6 and 7.

One embodiment described herein is a computer-implemented method, the method comprising: receiving, by one or more processors, data representative of one or more primer counts; identifying, by one or more processors, whether the data representative of any one of the one or more primer counts is suitable to be fit into a regression model; transferring into the regression model, by one or more processors, the data representative of the one or more suitable primer counts; determining, by one or more processors, one or more segments associated with the data representative of the one or more suitable primer counts by applying kernel change point detection to the data representative of the one or more suitable primer counts; and determining, by one or more processors, a fold change between the one or more segments relative to a baseline measure, wherein the fold change is representative of a quantity of unique molecules. In one aspect, the method further comprises: annotating, by one or more processors, the data representative of the one or more suitable primer counts with a value representative of an asymptote for the data. In another aspect, the method further comprises: removing by covariate correction, by one or more processors, all primer counts which are not suitable to be fit into the regression model; and applying guanine-cytosine (GC) bias correction, by one or more processors, to the asymptotes of the one or more suitable primer counts. In another aspect, the GC bias correction is locally weighted scatterplot smoothing (LOWESS). In another aspect, the regression model is a Michaelis-Menten (MM) model.

Another embodiment described herein is a computer program product, the computer program product comprising: one or more computer-readable storage media and program instructions stored on the one or more computer-readable storage media, the program instructions comprising: program instructions to receive data representative of one or more primer counts; program instructions to identify whether the data representative of any one of the one or more primer counts is suitable to be fit into a regression model; program instructions to transfer into the regression model the data representative of the one or more suitable primer counts; program instructions to determine one or more segments associated with the data representative of the one or more suitable primer counts by applying kernel change point detection to the data representative of the one or more suitable primer counts; and program instructions to determine a fold change between the one or more segments relative to a baseline measure, wherein the fold change is representative of a quantity of unique molecules. In one aspect, the program instructions further comprise: program instructions to annotate the data representative of the one or more suitable primer counts with a value representative of an asymptote for the data. In another aspect, the program instructions further comprise: removing by covariate correction, by one or more processors, all primer counts which are not suitable to be fit into the regression model; and applying guanine-cytosine (GC) bias correction, by one or more processors, to the asymptotes of the one or more suitable primer counts. In another aspect, the GC bias correction is locally weighted scatterplot smoothing (LOWESS). In another aspect, the regression model is a Michaelis-Menten (MM) model.

Another embodiment described herein is a computer system, the computer system comprising: one or more processors; one or more non-transitory computer-readable storage media; and program instructions stored on at least one of the one or more non-transitory computer-readable storage media for execution by at least one of the one or more processors, the program instructions comprising steps for implementing the following acts: program instructions to receive data representative of one or more primer counts; program instructions to identify whether the data representative of any one of the one or more primer counts is suitable to be fit into a regression model; program instructions to transfer into the regression model the data representative of the one or more suitable primer counts; program instructions to determine one or more segments associated with the data representative of the one or more suitable primer counts by applying kernel change point detection to the data representative of the one or more suitable primer counts; and program instructions to determine a fold change between the one or more segments relative to a baseline measure, wherein the fold change is representative of a quantity of unique molecules. The computer system of clause 11, the system further comprises: program instructions to annotate the data representative of the one or more suitable primer counts with a value representative of an asymptote for the data. In another aspect, the system further comprises: removing by covariate correction, by one or more processors, all primer counts which are not suitable to be fit into the regression model; and applying guanine-cytosine (GC) bias correction, by one or more processors, to the asymptotes of the one or more suitable primer counts. In another aspect, the GC bias correction is locally weighted scatterplot smoothing (LOWESS). In another aspect, the regression model is a Michaelis-Menten (MM) model.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A computer-implemented method, the method comprising:
   receiving, by one or more processors, data representative of one or more primer counts;
   identifying, by one or more processors, whether the data representative of any one of the one or more primer counts is suitable to be fit into a regression model;
   transferring into the regression model, by one or more processors, the data representative of the one or more suitable primer counts;
   determining, by one or more processors, one or more segments associated with the data representative of the one or more suitable primer counts by applying kernel change point detection to the data representative of the one or more suitable primer counts; and
   determining, by one or more processors, a fold change between the one or more segments relative to a baseline measure, wherein the fold change is representative of a quantity of unique molecules.
Clause 2. The computer-implemented method of clause 1, the method further comprising:
   annotating, by one or more processors, the data representative of the one or more suitable primer counts with a value representative of an asymptote for the data.
Clause 3. The computer-implemented method of clause 1 or 2, the method further comprising:
   removing by covariate correction, by one or more processors, all primer counts which are not suitable to be fit into the regression model; and
   applying guanine-cytosine (GC) bias correction, by one or more processors, to the asymptotes of the one or more suitable primer counts.
Clause 4. The computer-implemented method of any one of clauses 1-3, wherein the GC bias correction is locally weighted scatterplot smoothing (LOWESS).
Clause 5. The computer-implemented method of any one of clauses 1-4, wherein the regression model is a Michaelis-Menten (MM) model.
Clause 6. A computer program product, the computer program product comprising:
   one or more computer-readable storage media and program instructions stored on the one or more computer-readable storage media, the program instructions comprising:
      program instructions to receive data representative of one or more primer counts;
      program instructions to identify whether the data representative of any one of the one or more primer counts is suitable to be fit into a regression model;
      program instructions to transfer into the regression model the data representative of the one or more suitable primer counts;
      program instructions to determine one or more segments associated with the data representative of the one or more suitable primer counts by applying kernel change point detection to the data representative of the one or more suitable primer counts; and
      program instructions to determine a fold change between the one or more segments relative to a baseline measure, wherein the fold change is representative of a quantity of unique molecules.
Clause 7. The computer program product of clause 6, the program instructions further comprising:
   program instructions to annotate the data representative of the one or more suitable primer counts with a value representative of an asymptote for the data.
Clause 8. The computer program product of clause 6 or 7, the program instructions further comprising:
   removing by covariate correction, by one or more processors, all primer counts which are not suitable to be fit into the regression model; and
   applying guanine-cytosine (GC) bias correction, by one or more processors, to the asymptotes of the one or more suitable primer counts.
Clause 9. The computer program product of any one of clauses 6-8, wherein the GC bias correction is locally weighted scatterplot smoothing (LOWESS).
Clause 10. The computer program product of any one of clauses 6-9, wherein the regression model is a Michaelis-Menten (MM) model.
Clause 11. A computer system, the computer system comprising:
   one or more processors;
   one or more non-transitory computer-readable storage media; and
   program instructions stored on at least one of the one or more non-transitory computer-readable storage media for execution by at least one of the one or more processors, the program instructions comprising steps for implementing the following acts:
      program instructions to receive data representative of one or more primer counts;
      program instructions to identify whether the data representative of any one of the one or more primer counts is suitable to be fit into a regression model;
      program instructions to transfer into the regression model the data representative of the one or more suitable primer counts;
      program instructions to determine one or more segments associated with the data representative of the one or more suitable primer counts by applying kernel change point detection to the data representative of the one or more suitable primer counts; and
      program instructions to determine a fold change between the one or more segments relative to a baseline measure,
      wherein the fold change is representative of a quantity of unique molecules.
Clause 12. The computer system of clause 11, the system further comprising:
   program instructions to annotate the data representative of the one or more suitable primer counts with a value representative of an asymptote for the data.
Clause 13. The computer system of clause 11 or 12, the system further comprising:
   removing by covariate correction, by one or more processors, all primer counts which are not suitable to be fit into the regression model; and
   applying guanine-cytosine (GC) bias correction, by one or more processors, to the asymptotes of the one or more suitable primer counts.
Clause 14. The computer system of any one of clauses 11-13, wherein the GC bias correction is locally weighted scatterplot smoothing (LOWESS).
Clause 15. The computer system of any one of clauses 11-14, wherein the regression model is a Michaelis-Menten (MM) model.

### EXAMPLES

### Example 1

Copy Number Variant Detection without a Panel of Normals Using Anchored Multiplex PCR and Next Generation Sequencing

CNV methods that require normals, including the existing conventional Archer® Analysis CNV method, can produce different results for the same sample library depending on the choice of samples used for normalization. Input type, quality, read depth, and CNVs present in samples used for comparison can all impact the CNVs that can be detected in an unknown sample. Sourcing, preparing, and analyzing additional samples also increases the amount of work, time, cost to receive results.

In this example, raw reads from a sequenced Anchored Multiplex PCR (AMP) library were deduplicated to unique fragments using molecular barcodes incorporated during library preparation and aligned to the genome by Archer® Analysis as for any DNA workflow. Next, instead of using a paired normal or panel of normals to normalize counts, a modelling approach was used to account for the influence of common biases (GC %, PCR, sequencing biases, etc.) on observed counts for each primer in the sample. Suspected outliers were removed, then the remaining bias-corrected values proceeded to segmentation. The segmentation process groups together adjacent data points of similar value (i.e., copy number). The sample's baseline copy number was estimated from bias-corrected counts of autosomal primers within the sample. Fold change values were calculated relative to this estimated baseline copy number, rather than a paired normal or a panel of normals. Each segment's mean was tested against the estimated baseline copy number and p-values were reported.

### Example 2

### Comparison of Cohortless CNV Method to Current Archer Analysis CNV Method

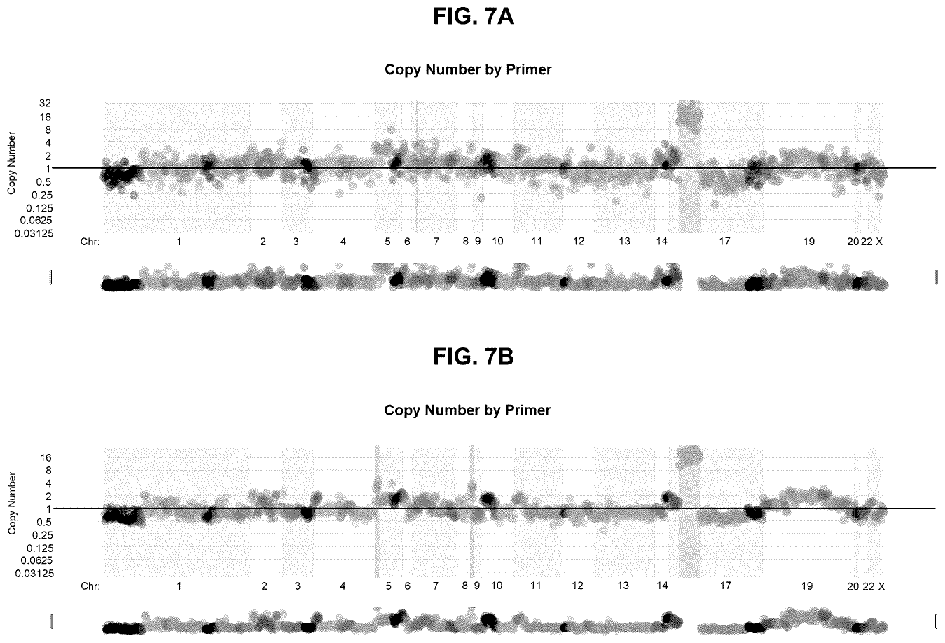

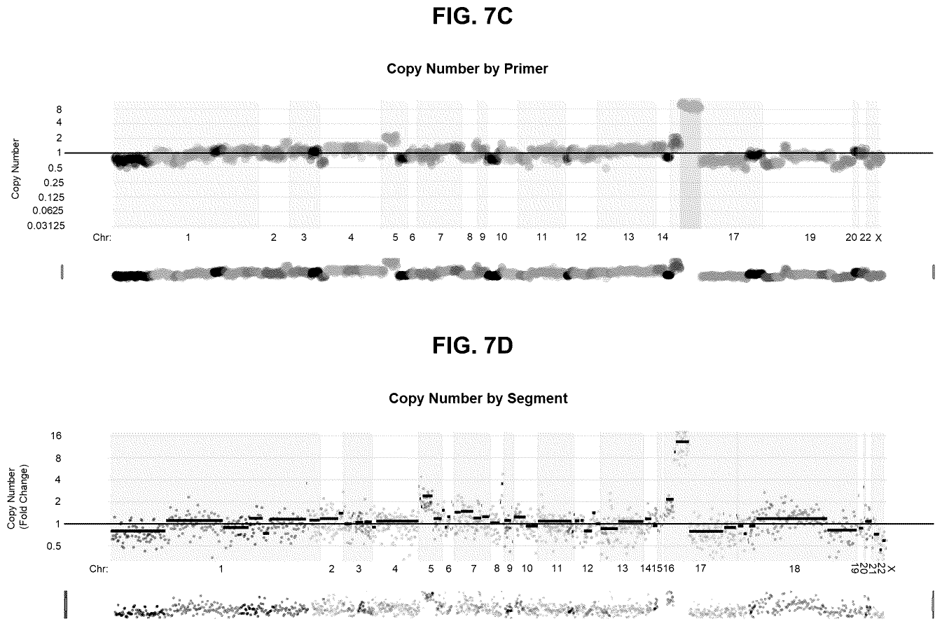

FIG. 7A-D show results of the conventional Archer® Analysis CNV method using three different cohorts as compared to the cohortless CNV method for the same library. 50 ng of DNA from FFPE tissue was prepared using the Archer® VARIANT Plex® Pan Solid Tumor panel. Each of FIG. 7A-D shows a cropped screenshot of the CNV results for this library using the conventional Archer® Analysis CNV method either using one of three different cohorts of samples (FIG. 7A-C) or a cohortless CNV method that does not require any external samples (FIG. 7D). In each plot, the various points represent primers. In FIG. 7D, the various black horizontal bars are CNV segments. In FIG. 7A, the panel of normals was made up of 7 unrelated normals—a mix of normal adjacent tissues, blood, and reference materials. In FIG. 7B, the normals consisted of 12 unrelated normal adjacent tissue samples, with no overlap with the normals used in FIG. 7A. FIG. 7C shows the results of using 39 unknown tumor samples as an approximated “normal” cohort. The results of the cohortless CNV method which uses only intrasample data for CNV detection are shown in FIG. 7D.

In FIG. 7A-C, the shaded vertical bars indicate events which passed the default filtering thresholds for the conventional method that requires a normal cohort. The visualization of the cohortless CNV method results shown in FIG. 7D does not include this shading.

A large ERBB2 gain, as well as a GNAS gain (not visible in the figures), were detected in each case, regardless of the CNV method or normal cohort used. Other than the ERBB2 gain, different genes are highlighted in each of FIG. 7A-C due to the different comparison cohorts. The results of the cohortless CNV method in FIG. 7D corroborated all the events highlighted by each of the current method analyses in FIGS. 7A-C, and also detected additional events which were not detectable or did not meet the thresholds of the current Archer® Analysis CNV method that requires normals.

### Example 3

### CNV Breakpoint Detection of Synthetic MET Amplification in Challenging Low Input Mass and Contrived Low Aberrant Cellularity Libraries

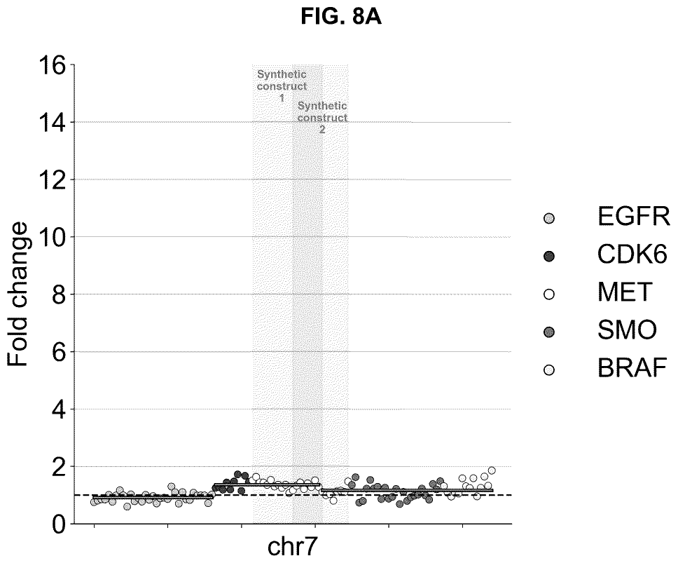

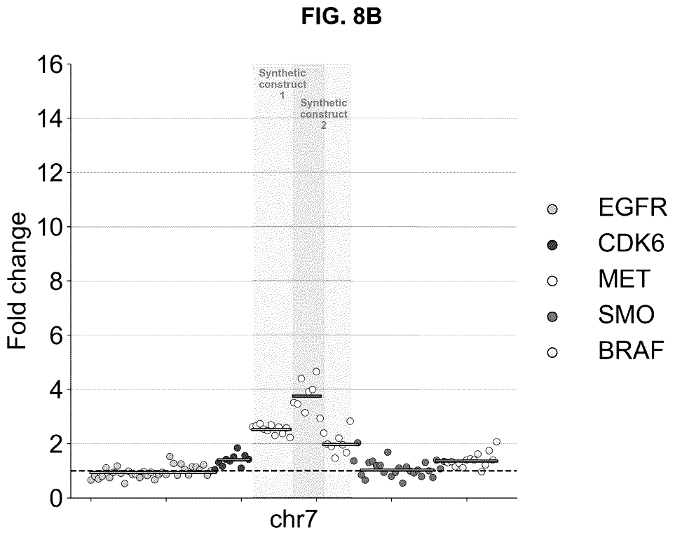

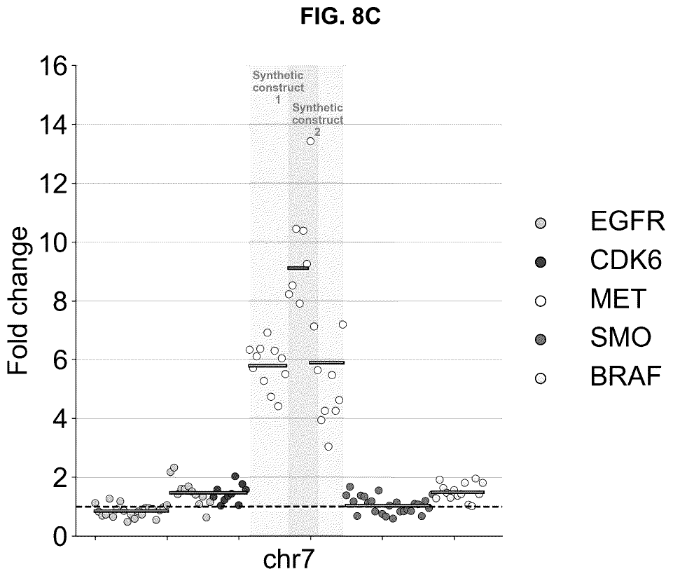

VARIANT Plex® libraries were prepared using 10 ng total input mass of either Seraseq® FFPE WT DNA reference material (FIG. 8A), Seraseq® Compromised FFPE Tumor DNA reference material (FIG. 8C), or a mix of 30% Tumor DNA reference material with the WT DNA reference material as background (FIG. 8B). Libraries were then sequenced and analyzed by the cohortless CNV process in Archer® Analysis.

Each data point in the plots represents a primer in the panel, shaded by the specific target gene (e.g., EGFR, CDK6, MET, SMO, or BRAF), and horizontal lines are drawn at the mean fold change of segments. The black dashed line in each plot represents the sample's baseline (fold change=1), against which primer and segment fold changes were calculated. The results shown are representative of both 10 ng replicates of each input and 50 ng replicates of the same.

The manufacturer used two overlapping synthetic constructs to create the MET gene amplification in the Seraseq® Compromised FFPE Tumor DNA reference material. The locations of these constructs are represented in each plot by the middle regions highlighted in gray. A greater amplification is expected where these constructs overlap (darker gray region). The cohortless CNV method was able to reliably and accurately detect the internal breakpoints that result from the overlap of these constructs, even in the 30% positive material mixed library at 10 ng (FIG. 8B), without requiring normal tissue.

### Example 4

Concordance of CNV Segment Fold Change Ratios with ddPCR Concentration Ratios

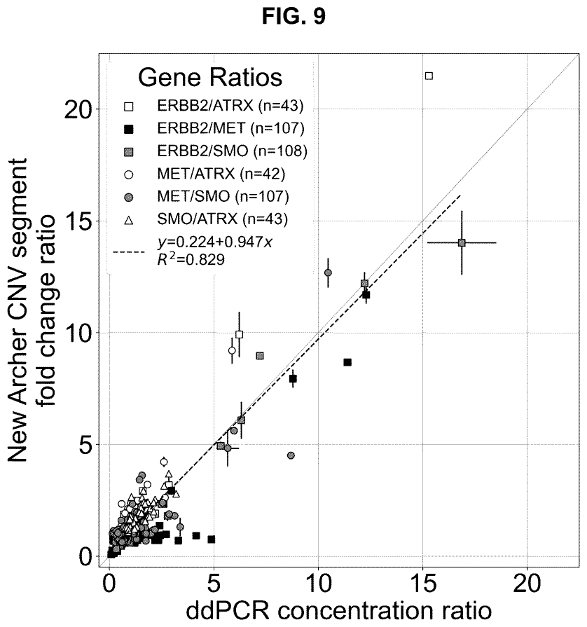

FIG. 9 shows the results of ddPCR and the cohortless CNV method for 108 unique inputs, including tumor FFPE extracts, cell lines, and reference materials, with a variety of amplifications and deletions. The various points are shaded according to the pair of genes that were compared.

The 108 unique inputs were assayed with 4 ddPCR probes located in MET, ERBB2, SMO, and ATRX. VARIANT Plex® libraries were prepared from the same 108 inputs using multiple panels, ranging in size from approximately 900 to 10,000 primers. Libraries were then sequenced and analyzed by the cohortless CNV process in Archer® Analysis.

The number of points for each gene ratio pair is unequal because of differences in panel content and genes covered, e.g., all panels covered ERBB2 and SMO, but some panels did not cover ATRX. Each point represents a ratio (or mean ratio when replicates from the same input material were available) between two genes calculated from ddPCR concentrations or CNV segment fold changes for segments that overlap with the coordinates of the ddPCR probes used. The data points are shaded according to the specific pair of genes compared. Standard deviation error bars are drawn for replicates of the same input material.

The agreement between the segment fold change ratios and the ddPCR concentration ratios indicated that the modeling and segmentation methods perform well across this set of inputs.

### Example 5

Selected Representative CNV Events Detected with Cohortless CNV Method

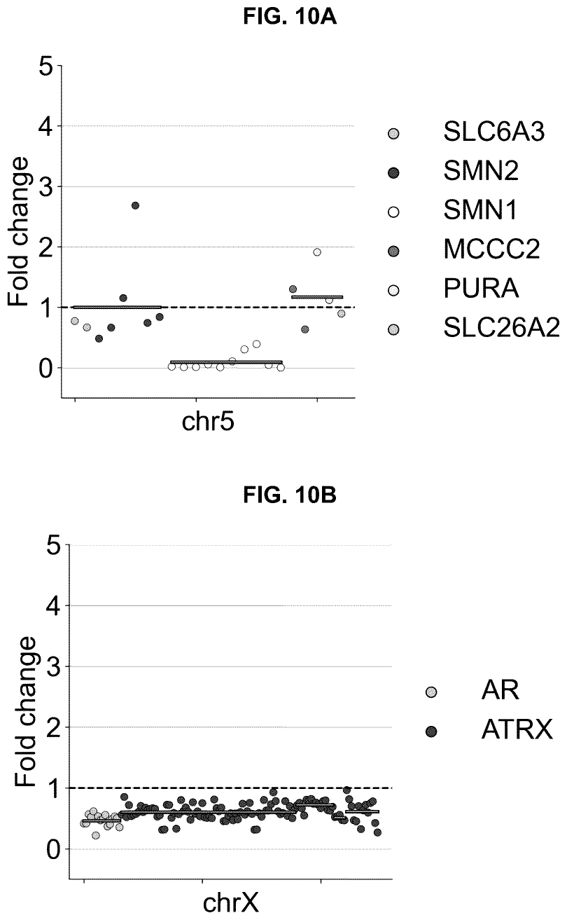

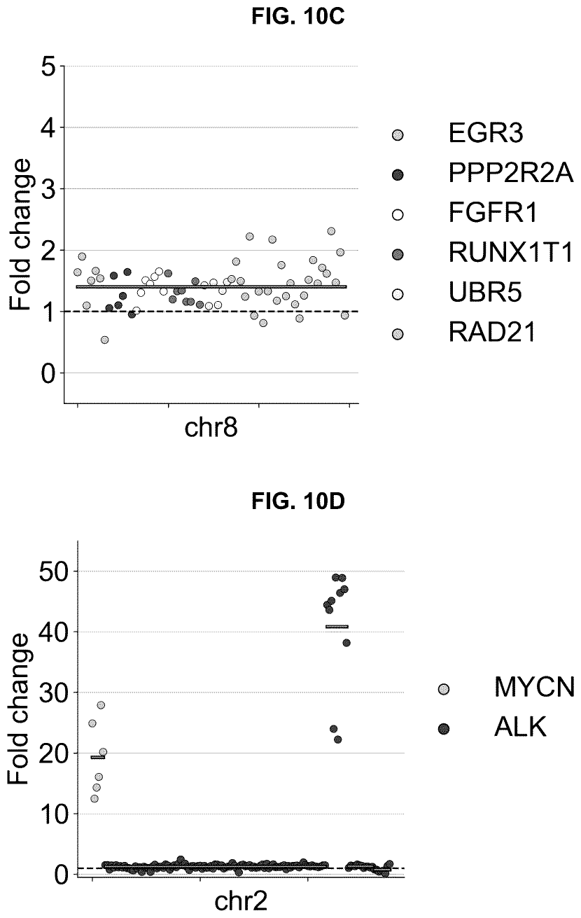

FIG. 10A-D show results demonstrating the capability of the cohortless CNV method to identify and call a variety of CNV events in different applications. In each figure plot, the points represent primers shaded by the specific gene they target. The dashed black line represents the baseline copy number (fold change=1). The dark gray lines represent the mean fold change for a segment. In FIG. 10A, a homozygous deletion of SMN2 on chromosome 5 was detected in a library prepared with 50 ng germline input with a small (˜500 primers) custom VARIANT Plex® panel. Heterozygous “losses” of X and Y in XY samples can also be observed, as shown in FIG. 10B. Furthermore, whole chromosome gains can be observed, as shown in FIG. 10C, which depicts a case of trisomy 8. In addition to being able to detect the above one or two copy number events, some of which span whole chromosomes, FIG. 10D depicts very large copy number gains detected in MYCN and exons 3 and 4 of ALK on chromosome 2 in a neuroblastoma cell line.

Overall, these results demonstrate that the cohortless CNV method, which does not use any paired or panel of normals, performs well on diverse input types, across a range of panel sizes, and can detect CNV events that vary in genomic span and fold change magnitude. As with any method, the specific size (in bp) resolution is dependent on the distribution of probes used (e.g., primers in the panel) and events of smaller copy number difference may be limited by input purity. The results further demonstrate the agreement of fold change measurements with ddPCR (FIG. 9), the ability of this method to accurately detect the breakpoints of sufficiently large gains in as little as 30% aberrant input (FIG. 8B), and the ability to detect CNV events as large as whole chromosomes (FIG. 10B-C) or as small as two exons (FIG. 10D). By performing CNV detection using this new cohortless method, it allows for reliable and accurate detection of CNVs without the overhead or confounding factors that comparisons to normals or a panel of normals can introduce.

### Example 6

### Performance of Various CNV Methods on VARIANTPlex Libraries

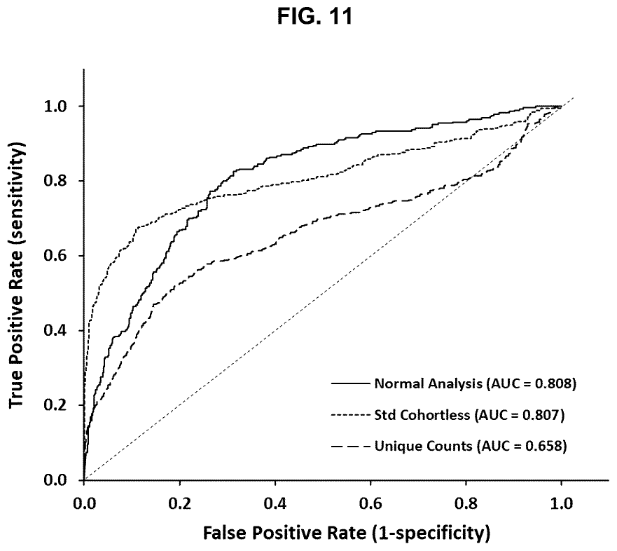

FIG. 11 shows a receiver operating characteristic (ROC) curve plot of sensitivity and specificity, demonstrating overall functionality of counts through the CNV process. These are compared to two internal NGS processes on the same data.

The ROC plot shows the performance of various CNV methods on 212 VARIANT Plex® libraries with a variety of expected CNV events. Input materials were primarily tumor FFPE, but also included fresh frozen tumor material, normal adjacent tissue, cell lines, and reference materials.

Libraries were prepared from those input materials using 5 different VARIANTPlex® panels ranging in size from 500 to 2600 primers. CNV calls in these libraries from the three methods depicted were compared to the expected CNV events and used to construct the ROC curve.

The “Normal Analysis” solid line of FIG. 11 represents the results of the existing conventional CNV method which requires subjects to create a normal cohort. The “Std Cohortless” small dashed line of FIG. 11 represents the results of the new cohortless CNV method which does not require a normal cohort or baseline reference. Instead, the cohortless CNV method models primer read accumulation and estimates the asymptote of a curve and then uses that asymptote as input into a segmentation and statistical process. The “Unique Counts” large dashed line of FIG. 11 differs by using non-modeled primer unique counts rather than the asymptote estimates as input into segmentation and statistical CNV detection. This “Unique Counts” method was included to show that the modeling of asymptotic primer counts provides a benefit over using the unique counts.

What is claimed:

1. A computer-implemented method, the method comprising:
receiving, by one or more processors, data representative of one or more primer counts;
identifying, by one or more processors, whether the data representative of any one of the one or more primer counts is suitable to be fit into a regression model;
transferring into the regression model, by one or more processors, the data representative of the one or more suitable primer counts;
determining, by one or more processors, one or more segments associated with the data representative of the one or more suitable primer counts by applying kernel change point detection to the data representative of the one or more suitable primer counts; and
determining, by one or more processors, a fold change between the one or more segments relative to a baseline measure,
wherein the fold change is representative of a quantity of unique molecules.

2. The computer-implemented method of claim 1, the method further comprising:
annotating, by one or more processors, the data representative of the one or more suitable primer counts with a value representative of an asymptote for the data.

3. The computer-implemented method of claim 2, the method further comprising:
removing by covariate correction, by one or more processors, all primer counts which are not suitable to be fit into the regression model; and
applying guanine-cytosine (GC) bias correction, by one or more processors, to the asymptotes of the one or more suitable primer counts.

4. The computer-implemented method of claim 3, wherein the GC bias correction is locally weighted scatterplot smoothing (LOWESS).

5. The computer-implemented method of claim 1, wherein the regression model is a Michaelis-Menten (MM) model.

6. A computer program product, the computer program product comprising:
one or more computer-readable storage media and program instructions stored on the one or more computer-readable storage media, the program instructions comprising:
program instructions to receive data representative of one or more primer counts;
program instructions to identify whether the data representative of any one of the one or more primer counts is suitable to be fit into a regression model;
program instructions to transfer into the regression model the data representative of the one or more suitable primer counts;
program instructions to determine one or more segments associated with the data representative of the one or more suitable primer counts by applying kernel change point detection to the data representative of the one or more suitable primer counts; and
program instructions to determine a fold change between the one or more segments relative to a baseline measure,
wherein the fold change is representative of a quantity of unique molecules.

7. The computer program product of claim 6, the program instructions further comprising:
program instructions to annotate the data representative of the one or more suitable primer counts with a value representative of an asymptote for the data.

8. The computer program product of claim 7, the program instructions further comprising:
removing by covariate correction, by one or more processors, all primer counts which are not suitable to be fit into the regression model; and
applying guanine-cytosine (GC) bias correction, by one or more processors, to the asymptotes of the one or more suitable primer counts.

9. The computer program product of claim 8, wherein the GC bias correction is locally weighted scatterplot smoothing (LOWESS).

10. The computer program product of claim 6, wherein the regression model is a Michaelis-Menten (MM) model.

11. A computer system, the computer system comprising:
one or more processors;
one or more non-transitory computer-readable storage media; and
program instructions stored on at least one of the one or more non-transitory computer-readable storage media for execution by at least one of the one or more processors, the program instructions comprising steps for implementing the following acts:
program instructions to receive data representative of one or more primer counts;
program instructions to identify whether the data representative of any one of the one or more primer counts is suitable to be fit into a regression model;
program instructions to transfer into the regression model the data representative of the one or more suitable primer counts;
program instructions to determine one or more segments associated with the data representative of the one or more suitable primer counts by applying kernel change point detection to the data representative of the one or more suitable primer counts; and
program instructions to determine a fold change between the one or more segments relative to a baseline measure,
wherein the fold change is representative of a quantity of unique molecules.

12. The computer system of claim 11, the system further comprising:
program instructions to annotate the data representative of the one or more suitable primer counts with a value representative of an asymptote for the data.

13. The computer system of claim 12, the system further comprising:
removing by covariate correction, by one or more processors, all primer counts which are not suitable to be fit into the regression model; and
applying guanine-cytosine (GC) bias correction, by one or more processors, to the asymptotes of the one or more suitable primer counts.

14. The computer system of claim 13, wherein the GC bias correction is locally weighted scatterplot smoothing (LOWESS).

15. The computer system of claim 11, wherein the regression model is a Michaelis-Menten (MM) model.
